(19) United States
(12) Patent Application Publication
(10) Pub. No.: US 2021/0194985 A1
(43) Pub. Date: Jun. 24, 2021

(54) TIMING CONTENT PRESENTATION BASED ON PREDICTED RECIPIENT MENTAL STATE

(71) Applicant: AT&T Intellectual Property I, L.P., Atlanta, GA (US)

(72) Inventors: Natalie Gilbert, Warren, NJ (US); Mazin Gilbert, Warren, NJ (US)

(21) Appl. No.: 16/723,382

(22) Filed: Dec. 20, 2019

(51) Int. Cl. H04L 29/08; H04W 4/029; G06K 9/00; G06K 9/46; G10L 25/63; G10L 15/22; G06N 20/00; G06Q 30/02; A61B 5/021; A61B 5/00; A61B 5/024; A61B 5/11; A61B 5/16

(52) U.S. Cl. CPC H04L 67/32; H04W 4/029; H04L 67/22; G06K 9/00362; G06K 9/46; G10L 25/63; A61B 5/165; G06N 20/00; G06Q 30/0201; A61B 5/021; A61B 5/0022; A61B 5/024; A61B 5/1118; G10L 15/22

(57) ABSTRACT

Content presentation is timed based on an individual's predicted mental state. In one example, a method performed by a processing system includes extracting a feature set from data that is collected by at least one sensor, where the sensor is monitoring an individual, the feature set comprises at least one feature of the individual, and the feature set excludes features extracted from images of the individual, predicting a current mental state of the individual, where the current mental state is predicted by providing the feature set as input to a machine learning model, sending media content to an endpoint device of the individual when the current mental state indicates that the individual is likely to be receptive to receiving the media content, and postponing sending media content to the endpoint device when the current mental state indicates that the individual is unlikely to be receptive to receiving the media content.

The present disclosure relates generally to just-in-time presentation of content, and relates more particularly to devices, non-transitory computer-readable media, and methods for timing content presentation based on the intended recipient's predicted mental state.

### BACKGROUND

As mobile devices become more powerful and more ubiquitous, the presentation of media content, and in particular advertising, is shifting towards a just-in-time model. That is, presentation of content on a recipient's mobile device may be opportunistically timed to increase engagement of the recipient with the content. For instance, a coupon for a discount at a coffee shop may be texted, emailed, and/or sent as an in-application notification to the mobile device of an individual as the individual is walking past the coffee shop.

### BRIEF DESCRIPTION OF THE DRAWINGS

The teachings of the present disclosure can be readily understood by considering the following detailed description in conjunction with the accompanying drawings, in which:

FIG. 1 illustrates an example network related to the present disclosure;

FIG. 2 illustrates a flowchart of a method for timing the presentation content to an individual based on the individual's predicted mental state, in accordance with the present disclosure;

FIG. 3 illustrates an example matrix that may be constructed in order to track an individual's features over various intervals of time; and

FIG. 4 depicts a high-level block diagram of a computing device specifically programmed to perform the functions described herein.

To facilitate understanding, identical reference numerals have been used, where possible, to designate identical elements that are common to the figures.

### DETAILED DESCRIPTION

In one example, the present disclosure improves the engagement of an individual with media content by timing presentation of the content to the individual based on the individual's predicted mental state. In particular, the individual's mental state can be predicted through minimally intrusive means in order to detect when the individual is likely to be most emotionally and psychologically receptive to receiving content. In one example, a method performed by a processing system in a telecommunications network includes extracting a feature set from data that is collected by at least one sensor, wherein the at least one sensor is monitoring an individual, wherein the feature set comprises at least one feature of the individual, and wherein the feature set excludes features extracted from images of the individual, predicting a current mental state of the individual, wherein the current mental state is predicted by providing the feature set as an input to a machine learning model, sending media content to an endpoint device of the individual when the current mental state of the individual indicates that the individual is likely to be receptive to receiving the media content, and postponing sending media content to the endpoint device of the individual when the current mental state of the individual indicates that the individual is unlikely to be receptive to receiving the media content.

In another example, a non-transitory computer-readable medium stores instructions which, when executed by a processing system including at least one processor, cause the processing system to perform operations. The operations include extracting a feature set from data that is collected by at least one sensor, wherein the at least one sensor is monitoring an individual, wherein the feature set comprises at least one feature of the individual, and wherein the feature set excludes features extracted from images of the individual, predicting a current mental state of the individual, wherein the current mental state is predicted by providing the feature set as an input to a machine learning model, sending media content to an endpoint device of the individual when the current mental state of the individual indicates that the individual is likely to be receptive to receiving the media content, and postponing sending media content to the endpoint device of the individual when the current mental state of the individual indicates that the individual is unlikely to be receptive to receiving the media content.

In another example, a device includes a processing system including at least one processor and a non-transitory computer-readable medium that stores instructions which, when executed by the processing system, cause the processing system to perform operations. The operations include extracting a feature set from data that is collected by at least one sensor, wherein the at least one sensor is monitoring an individual, wherein the feature set comprises at least one feature of the individual, and wherein the feature set excludes features extracted from images of the individual, predicting a current mental state of the individual, wherein the current mental state is predicted by providing the feature set as an input to a machine learning model, sending media content to an endpoint device of the individual when the current mental state of the individual indicates that the individual is likely to be receptive to receiving the media content, and postponing sending media content to the endpoint device of the individual when the current mental state of the individual indicates that the individual is unlikely to be receptive to receiving the media content.

As discussed above, the presentation of media content, and in particular advertising, is shifting toward a just-in-time model, especially as mobile devices become more powerful and more ubiquitous. For instance, presentation of content on a recipient's mobile device may be opportunistically timed to increase engagement of the recipient with the content. As an example, a coupon for a discount at a coffee shop may be texted, emailed, and/or sent as an in-application notification to the mobile device of an individual as he is walking past the coffee shop. Thus, the individual receives the content (i.e., the coupon) at the time at which the content is most useful to him (i.e., when he is at or near the coffee shop). As such, the just-in-time model is beneficial to the content creator (who may see increased engagement with the content) as well as to the content recipient (who may receive useful information at opportune times).

However, the just-in-time model may also run the risk of presenting too much content to individuals, which can backfire. For instance, if an individual receives what he considers to be too much content (e.g., x number of emails in an hour, in a day, or the like), or if the individual receives the content at a time when he is not emotionally or psychologically receptive to the content (e.g., the individual is in a hurry, is working, or is trying to concentrate on something), then the individual may begin to ignore content that he would, under different circumstances, have engaged with. Although some advertising systems may attempt to predict an individual's likely emotional reaction to a specific item of advertising content (e.g., should an advertisement with a sad tone be sent to an individual who already appears to be sad?), such systems typically do not seek to determine whether the individual's present emotional or psychological state is conducive to receiving advertising content in general. Moreover, most approaches to predicting an individual's emotional state often rely on the analysis of real time or recent images of the individual's face and/or samples of the individual's voice. However, as concerns over consumer privacy mount, many individuals may find such approaches to be intrusive and uncomfortable.

Examples of the present disclosure predict the current mental (e.g., emotional and/or psychological) state of a candidate recipient of media content (e.g., an individual to whom a system may be considering sending an item of media content) based on information such as the candidate recipient's current health, purchasing habits, context, and personal features. Thus, the candidate recipient's current mental state may be predicted in an unobtrusive manner, e.g., without relying on intrusive approaches such as facial or voice analysis. The current mental state of the candidate recipient may be used to infer a general receptiveness of the candidate recipient to media content at the current time (e.g., is the candidate recipient likely to engage with an advertisement if the advertisement is sent right now?), irrespective of the actual contents (e.g., subject matter, tone, etc.) of the media content. Thus, the sending of media content to the candidate recipient may be timed to coincide with times at which the candidate recipient is deemed most receptive to (and therefore more likely to engage with) the media content. Conversely, sending of media content to the candidate recipient may be avoided at times when the candidate recipient is deemed unlikely to be receptive to (and therefore less likely to engage with) the media content. Over time, therefore, the candidate recipient may experience less fatigue with respect to the just-in-time content.

Although examples of the present disclosure may be described within the example context of sending commercial media content (e.g., coupons, advertisements, and the like), it will be understood that the examples disclosed herein could also be used to send media content that is less overtly commercial, or is not commercial at all (e.g., entertainment content, social media updates from friends, etc.).

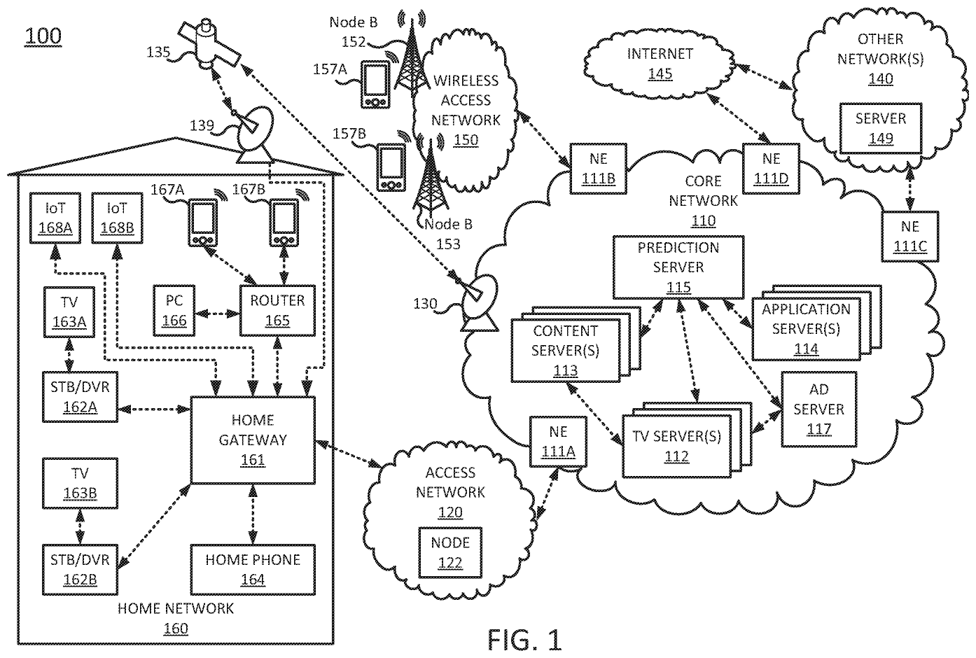

To better understand the present disclosure, FIG. 1 illustrates an example network 100 related to the present disclosure. As shown in FIG. 1, the network 100 connects mobile devices 157A, 157B, 167A and 167B, and home network devices such as home gateway 161, set-top boxes (STBs) 162A, and 162B, television (TV) 163A and TV 163B, home phone 164, router 165, personal computer (PC) 166, and so forth, with one another and with various other devices via a core network 110, a wireless access network 150 (e.g., a cellular network), an access network 120, other networks 140 and/or the Internet 145.

In one example, wireless access network 150 comprises a radio access network implementing such technologies as: global system for mobile communication (GSM), e.g., a base station subsystem (BSS), or IS-95, a universal mobile telecommunications system (UMTS) network employing wideband code division multiple access (WCDMA), or a CDMA3000 network, among others. In other words, wireless access network 150 may comprise an access network in accordance with any “second generation” (2G), “third generation” (3G), “fourth generation” (4G), Long Term Evolution (LTE) or any other yet to be developed future wireless/cellular network technology including “fifth generation” (5G) and further generations. While the present disclosure is not limited to any particular type of wireless access network, in the illustrative example, wireless access network 150 is shown as a UMTS terrestrial radio access network (UTRAN) subsystem. Thus, elements 152 and 153 may each comprise a Node B or evolved Node B (eNodeB).

In one example, each of mobile devices 157A, 157B, 167A, and 167B may comprise any subscriber/customer endpoint device configured for wireless communication such as a laptop computer, a Wi-Fi device, a Personal Digital Assistant (PDA), a mobile phone, a smartphone, an email device, a computing tablet, a messaging device, a wearable smart device (e.g., a smart watch or fitness tracker), a gaming console, and the like. In one example, any one or more of mobile devices 157A, 157B, 167A, and 167B may have both cellular and non-cellular access capabilities and may further have wired communication and networking capabilities.

As illustrated in FIG. 1, network 100 includes a core network 110. In one example, core network 110 may combine core network components of a cellular network with components of a triple play service network; where triple play services include telephone services, Internet services and television services to subscribers. For example, core network 110 may functionally comprise a fixed mobile convergence (FMC) network, e.g., an IP Multimedia Subsystem (IMS) network. In addition, core network 110 may functionally comprise a telephony network, e.g., an Internet Protocol/Multi-Protocol Label Switching (IP/MPLS) backbone network utilizing Session Initiation Protocol (SIP) for circuit-switched and Voice over Internet Protocol (VoIP) telephony services. Core network 110 may also further comprise a broadcast television network, e.g., a traditional cable provider network or an Internet Protocol Television (IPTV) network, as well as an Internet Service Provider (ISP) network. The network elements 111A-111D may serve as gateway servers or edge routers to interconnect the core network 110 with other networks 140 (which may include servers 149), Internet 145, wireless access network 150, access network 120, and so forth. As shown in FIG. 1, core network 110 may also include a plurality of television (TV) servers 112, a plurality of content servers 113, a plurality of application servers 114, an advertising server (AS) 117, and a prediction server 115 (e.g., an application server). For ease of illustration, various additional elements of core network 110 are omitted from FIG. 1.

With respect to television service provider functions, core network 110 may include one or more television servers 112 for the delivery of television content, e.g., a broadcast server, a cable head-end, and so forth. For example, core network 110 may comprise a video super hub office, a video hub office and/or a service office/central office. In this regard, television servers 112 may interact with content servers 113, advertising server 117, and prediction server 115 to select which video programs, or other content and advertisements to provide to the home network 160 and to others.

In one example, content servers 113 may store scheduled television broadcast content for a number of television channels, video-on-demand programming, local programming content, gaming content, and so forth. The content servers 113 may also store other types of media that are not audio/video in nature, such as audio-only media (e.g., music, audio books, podcasts, or the like) or video-only media (e.g., image slideshows). For example, content providers may upload various contents to the core network to be distributed to various subscribers. Alternatively, or in addition, content providers may stream various contents to the core network for distribution to various subscribers, e.g., for live content, such as news programming, sporting events, and the like. In one example, advertising server 117 stores a number of advertisements that can be selected for presentation to viewers, e.g., in the home network 160 and at other downstream viewing locations. For example, advertisers may upload various advertising content to the core network 110 to be distributed to various viewers.

In one example, prediction server 115 may generate a prediction as to whether or not a current time is an opportune time to send content (e.g., advertisements, coupons, or other content) to a particular individual. In one example, the prediction is based on a predicted mental (e.g., emotional and/or psychological) state of the particular individual.

For instance, in one example, the prediction server 115 may collect data from a plurality of sensors, where the plurality of sensors may include sensors that are located in proximity to the individual. The prediction server may extract, from this data, a plurality of features that may be indicative of the individual's current mental state. For instance, if the individual is wearing a fitness tracker, the prediction server 115 may be able to extract the individual's heart rate, blood pressure, steps walked, stairs climbed, other exercise activity (e.g., time spent exercising, type of exercise, etc.), amount of time spent sleeping the previous night, symptoms of current illnesses or health conditions, and/or other health indicators over various intervals of time. If the user is carrying a mobile phone, the prediction server may be able to extract the individual's location (which, in turn, may allow the prediction server 115 to identify nearby businesses), information about the individual's recent activities and/or purchases (e.g., through email receipts received on the mobile phone, text alerts from the individual's bank), information about applications currently in use by the individual (e.g., a destination the individual is being guided to by a navigation application, a movie or genre of movie the individual is watching via a streaming video application, a genre of music the individual is listening to on a streaming music application, etc.), and/or other indicators of the individual's context over various intervals of time.

The prediction server 115 may analyze a combination of these features at various intervals of time and may, from the analysis of the features, predict a current mental state of the individual. In one example, the feature data may be supplemented by the individual's history (e.g., mental states under which the individual was observed to have made purchase in the past, mental states under which the individual was observed to have interacted with content in the past, etc.) and/or preferences (e.g., times at which the individual prefers to receive or not receive content).

Based on the individual's predicted current mental state, the prediction server 115 may further predict whether the current time is an opportune time to send media content (e.g., advertisements, coupons, and/or other content) to the individual's endpoint device. In one example, an opportune time is a time at which the individual is predicted to be emotionally and psychologically receptive to receiving content. In one example, the individual may be assumed to be receptive to receiving content when the individual is happy and may be assumed to be not receptive to receiving content when the individual is unhappy. However, the granularity of the analysis of the individual's mental state and the correlations between mental state and receptiveness may be greater in other examples.

In some examples, the prediction server 115 may monitor the individual's responses to content in order to better understand the mental states under which the individual is most receptive and/or unreceptive to content. This may help the prediction server 115 to fine tune its future predictions.

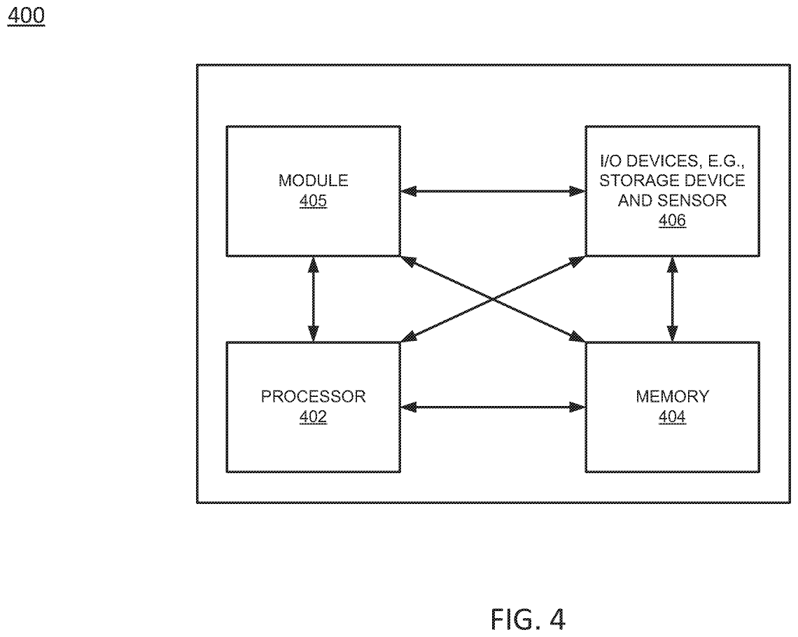

In one example, any or all of the television servers 112, content servers 113, application servers 114, prediction server 115, and advertising server 117 may comprise a computing system, such as computing system 400 depicted in FIG. 4.

In one example, the access network 120 may comprise a Digital Subscriber Line (DSL) network, a broadband cable access network, a Local Area Network (LAN), a cellular or wireless access network, a 3^{rd} party network, and the like. For example, the operator of core network 110 may provide a cable television service, an IPTV service, or any other type of television service to subscribers via access network 120. In this regard, access network 120 may include a node 122, e.g., a mini-fiber node (MFN), a video-ready access device (VRAD) or the like. However, in another example node 122 may be omitted, e.g., for fiber-to-the-premises (FTTP) installations. Access network 120 may also transmit and receive communications between home network 160 and core network 110 relating to voice telephone calls, communications with web servers via the Internet 145 and/or other networks 140, and so forth.

Alternatively, or in addition, the network 100 may provide television services to home network 160 via satellite broadcast. For instance, ground station 130 may receive television content from television servers 112 for uplink transmission to satellite 135. Accordingly, satellite 135 may receive television content from ground station 130 and may broadcast the television content to satellite receiver 139, e.g., a satellite link terrestrial antenna (including satellite dishes and antennas for downlink communications, or for both downlink and uplink communications), as well as to satellite receivers of other subscribers within a coverage area of satellite 135. In one example, satellite 135 may be controlled and/or operated by a same network service provider as the core network 110. In another example, satellite 135 may be controlled and/or operated by a different entity and may carry television broadcast signals on behalf of the core network 110.

In one example, home network 160 may include a home gateway 161, which receives data/communications associated with different types of media, e.g., television, phone, and Internet, and separates these communications for the appropriate devices. The data/communications may be received via access network 120 and/or via satellite receiver 139, for instance. In one example, television data is forwarded to set-top boxes (STBs)/digital video recorders (DVRs) 162A and 162B to be decoded, recorded, and/or forwarded to television (TV) 163A and TV 163B for presentation. Similarly, telephone data is sent to and received from home phone 164; Internet communications are sent to and received from router 165, which may be capable of both wired and/or wireless communication. In turn, router 165 receives data from and sends data to the appropriate devices, e.g., personal computer (PC) 166, mobile devices 167A and 167B, and so forth. In one example, router 165 may further communicate with TV (broadly a display) 163A and/or 163B, e.g., where one or both of the televisions is a smart TV. In one example, router 165 may comprise a wired Ethernet router and/or an Institute for Electrical and Electronics Engineers (IEEE) 802.11 (Wi-Fi) router, and may communicate with respective devices in home network 160 via wired and/or wireless connections.

IoT devices 168A and 168B may include any types of devices that are capable of being controlled automatically and/or remotely. For instance, the IoT devices 168A and 168B may include “smart” home devices, such as a smart thermostat, a smart lighting system, or the like. The IoT devices 168A and 168B may also include gaming devices, such as gaming controllers, a gaming chair, or the like. Although FIG. 1 illustrates two IoT devices 168A and 168B, it will be appreciated that the home network 160 may include any number of IoT devices. A greater number and variety of IoT devices may allow for a more accurate estimate of an individual's current mental state to be generated.

Those skilled in the art will realize that the network 100 may be implemented in a different form than that which is illustrated in FIG. 1, or may be expanded by including additional endpoint devices, access networks, network elements, application servers, etc. without altering the scope of the present disclosure. For example, core network 110 is not limited to an IMS network. Wireless access network 150 is not limited to a UMTS/UTRAN configuration. Similarly, the present disclosure is not limited to an IP/MPLS network for VoIP telephony services, or any particular type of broadcast television network for providing television services, and so forth.

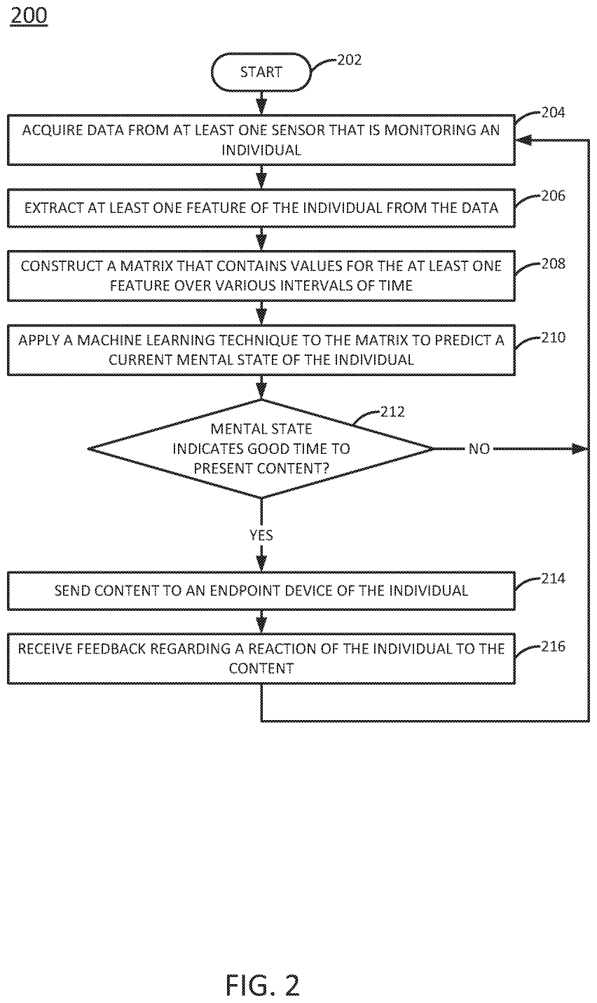

To further aid in understanding the present disclosure, FIG. 2 illustrates a flowchart of a method 200 for timing the presentation content to an individual based on the individual's predicted mental state, in accordance with the present disclosure. In one example, the method 200 may be performed by a prediction server that is configured to time content presentation based on the intended recipient's predicted mental state, such as the prediction server 115 illustrated in FIG. 1. However, in other examples, the method 200 may be performed by another device, such as the processor 402 of the system 400 illustrated in FIG. 4. For the sake of example, the method 200 is described as being performed by a processing system.

The method 200 begins in step 202. In step 204, the processing system may acquire data from at least one sensor that is monitoring an individual. The sensors that collect the data may comprise sensors that are integrated into the individual's mobile device (e.g., mobile phone, tablet computer, portable gaming system, or the like), into the individual's wearable devices (e.g., fitness tracker, smart watch, smart glasses, etc.), into IoT devices that may be located close enough to the individual to detect data about the individual (e.g., a smart bike lock or Bluetooth tracker, a connected security system, a smart speaker connected to a virtual assistant, etc.), or into other devices.

In one example, the processing system may also be integrated into the device into which the sensors are integrated. For instance, the processing system may be the processing system of the individual's mobile phone. In this case, the processing system may acquire the data directly from the local storage of the device. However, in other examples, the processing system may be integrated into a device that is remotely located from the device into which the sensors are integrated. For instance, the processing system may be the processing system of an application server, and the device into which the sensors are integrated may communicate with the application server over a telecommunications network. In this case, the processing system may receive the data in one or more packets that are transmitted in signals sent by the device into which the sensors are integrated.

In one example, the data acquired in step 202 excludes images of the individual and voice samples of the individual. That is, the data acquired in step 202 comprises data other than images (e.g., facial images) and voice samples of the individual. For instance, in one example, the data acquired in step 202 may comprise one or more of: the individual's health data (e.g., as collected by the individual's mobile phone or wearable fitness tracker), the individual's recent purchasing history (e.g., as collected by a wallet application or an email application operating on the individual's mobile phone), the individual's current context (e.g., as collected by a location sensor or global positioning system application operating on the individual's mobile phone or wearable fitness tracker), and/or the individual's personal preferences (e.g. as may be stored in an application operating on the individual's mobile phone). Thus, the data acquired in step 202 may comprise readings from various different types of sensors.

In step 206, the processing system may extract a feature set including at least one feature of the individual from the set of data. In one example, the feature set excludes features extracted from images or voice samples of the individual, as discussed above (even if images and voice samples are available). In one example, feature(s) extracted in step 206 may describe the individual's behavior and/or preferences over the defined interval of time. For instance, a feature extracted from the individual's health data may include the individual's average blood pressure over the interval of time, average heart rate over the interval of time, number of steps walked or stairs climbed over the interval of time, average rate of speed over the interval of time (e.g., miles per hour, steps per minute etc.), average stride length over the interval of time, activity type during the interval of time (e.g., walking, running, climbing), amount of time spent exercising, amount of time spent sleeping, symptoms of current illnesses or health conditions, and other features.

A feature extracted from the individual's recent purchasing history data may include the time of the individual's last purchase, the number of purchases made by the individual over the interval of time, the nature of any purchases made by the individual over the interval of time (e.g., what the individual purchased during the interval of time), whether any advertisements were presented to the individual prior to any purchases made over the interval of time, the nature of any advertisements presented to the individual over the interval of time (e.g., amount of discounts, types of products or services, etc.), a ratio of the number of content items the individual responded to over the interval of time (e.g., by making a purchase, by reviewing or otherwise interacting with the content items, etc.) to a number of content items presented to the individual over the interval period of time (e.g., a history of the individual's interaction with content items that have been presented to the individual), and other features.

A feature extracted from the individual's current context data may include a center of the individual's location over the interval of time (e.g., global positioning system coordinates, longitude and latitude, location name, etc.), businesses located within a defined radius of the individual's location over the interval of time (e.g., within a number of blocks, miles, or the like), the individual's companions over the interval of time (e.g., children, coworkers, friends, or the like), information about applications in use by the individual over the interval of time (e.g., a destination the individual is being guided to by a navigation application, a movie or genre of movie the individual is watching via a streaming video application, a genre of music the individual is listening to on a streaming music application, etc.), and other data.

A feature extracted from the individual's personal preference data may include the individual's demographic data (e.g., age, gender, marital status, etc.) and any preferences relating to content (e.g., what types of content the individual prefers to receive or not receive, times of day at which the individual prefers to receive or not receive content, etc.). In one example, the demographic data and preference data may be stored in a location that is accessible to the processing system (e.g., on the individual's device or in a remote database or application server). The demographic and preference data may be explicitly provided by the individual (e.g., in a profile created by the individual) or may be learned through observation of the individual (e.g., using machine learning).

As discussed above, the features extracted in step 206 may comprise features exhibited by the individual over a defined interval of time (e.g., over the last x seconds, over the last x minutes, etc.). Moreover, step 206 may result in the extraction of a plurality of features (e.g., up to twenty features, more than twenty features, etc.). The plurality of features may include any combination of the above-described features, but does not necessarily include every feature described above. Additionally, the plurality of features may include features not explicitly described above.

In step 208, the processing system may construct (or update, if the processing system is not performing the first iteration of the method 200) a matrix of the feature(s) extracted in step 206 over various intervals of time. Thus, the matrix may contain feature values for the individual as measured over various intervals of time during which the individual was monitored.

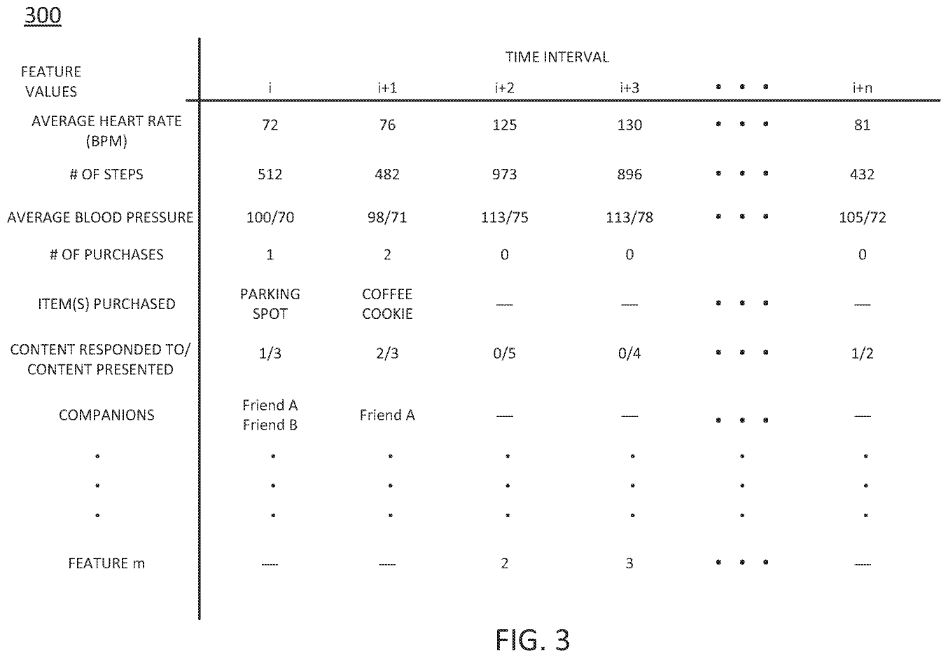

FIG. 3, for instance, illustrates an example matrix 300 that may be constructed in order to track an individual's features over various intervals of time. As illustrated, the rows of the example matrix 300 may define m different features of an individual, including an average heart rate, a number of steps, an average blood pressure, a number of purchases made, items purchased, a ratio of a number of items of content to which the individual responded to a number of items of content presented to the individual, the individual's companions, and other features. As discussed above, in one example, m may be a number up to twenty. In another example, m may be a number exceeding twenty.

The columns of the example matrix may define n+1 different intervals of time (e.g., i through i+n) during which the features of the individual may have been extracted from sensor data. In one example, each of the intervals i through i+n are of equal lengths (e.g., feature values may be extracted every x minutes, every x seconds, or the like, where each interval represents an interval of x minutes, x seconds, or the like). In other words, the features may be extracted at regular intervals. In another example, however, at least two of the intervals i through i+n are of different lengths. In other words, the features may be extracted at irregular intervals (e.g., at random intervals, at intervals that are triggered in response to specific events, etc.).

At each intersection of the example matrix 300 (e.g., each meeting of one row with one column), a value may be recorded to indicate a state of the corresponding feature during the corresponding interval. For instance, according to the example matrix 300, the individual walked 973 steps during the interval i+2 and had an average heart rate of 125 beats per minute (bpm) during the same time.

Referring back to FIG. 2, in step 210, the processing system may apply a machine learning model to the matrix to predict the individual's current mental state. In other words, the feature set for the individual (as defined by the matrix) is provided as an input to a machine learning model, where the output of the machine leaning model is the predicted current mental state of the individual. In one example, the machine learning model may utilize a convolutional deep learning technique in order to make the prediction, where the convolutional deep learning technique may be trained on a large set of training data. The training data may include values for various features (corresponding to either the specific individual or to a group of individuals) as well as some conclusion about the mental state corresponding to the various features (e.g., happy or unhappy, whether a purchase was made or not). In other examples, other types of machine learning techniques (including, for instance, support vector machines) may be applied in step 210 in addition to or instead of convolutional deep learning.

In one example, the output of the machine learning model is a binary decision. For instance, the machine learning model may apply an algorithm that combines the various features in the matrix in order to generate a score. If the score is below a predefined threshold, the individual may be assumed to be unhappy; if the score is above the predefined threshold, the individual may be assumed to be happy. Thus, in this example, the score may function as a predicted likelihood that the individual is happy. In other examples, however, the machine learning model may predict a mental state that falls within a broader (e.g., non-binary) range of mental states (e.g., happy, unhappy, scared, in a hurry, tired, etc.). In other examples, the output of the machine learning model may simply comprise a confidence that the individual's current mental state is of a particular nature (e.g., x percent confidence that the individual is happy).

In step 212, the processing system may determine whether the individual's predicted current mental state indicates that it is a good time to present content to the individual. In one example, the predicted current mental state may be correlated to a likelihood that indicates whether the individual is likely to be receptive to receiving content at the current time. For instance, the processing system may determine (e.g., based on prior observation of the individual, based on prior observation of other individuals, or based on the individual's preferences) that the individual is likely to interact with content (e.g., to view the content, to make a purchase in response to the content, etc.) when he is happy, but is unlikely to interact with content when he is unhappy. To improve the precision of this determination, reinforcement learning techniques may be continuously applied in order to adapt to the individual's preferences.

If the processing system determines in step 212 that content should not be presented to the individual at the current time, then the method 200 may return to step 204, and the processing system may continue to acquire data from at least one sensor and to process the data as described in connection with steps 206-212. Thus, the processing system postpones sending content to the individual, at least temporarily (e.g., at least while the individual's predicted current mental state indicates that the individual is unlikely to be receptive to the content).

If, however, the processing system determines in step 212 that content should be presented to the individual at the current time, then the method 200 may proceed to step 214, and the processing system may send content (e.g., via the telecommunications network) to an endpoint device of the individual. In one example, the endpoint device is an endpoint device that is currently on the individual's person (e.g., a mobile phone, a smart watch, smart glasses, a fitness tracker, or the like) as opposed to an endpoint device that is not currently on the individual's person (e.g., a home computer, a smart television, a set top box, or the like). For instance, the endpoint device to which the content is sent may be one of the endpoint devices from which data was acquired in step 204.

In one example, the content may comprise text, an image, an audio recording, a video, a machine readable code, or other types of content. In one example, the content may be delivered to an email application or a text messaging application on the individual's endpoint device. In this case, an email or a text message may contain an embedded copy of the content, an attachment containing the content, or a hyperlink to the content (e.g., which may be opened in a separate application, such as a web browser application). In another example, the content may be delivered to a dedicated application on the individual's endpoint device, such as an application that collects coupons. In this case, an alert may be sent via email, text-message, or an in-application alert to notify the individual that content is available for view.

In one example, the tone or subject matter of the content may not be correlated to the individual's current mental state. That is, the individual's current mental state may affect whether or not content is sent, but may not affect the actual nature of the content. In one example, the nature of the content may instead be dependent on the individuals' current location (e.g., if the individual is within x feet of a coffee shop and his current mental state indicates that he is receptive to receiving content, send him a coupon for the coffee shop). In this way, the method 200 may be considered content-agnostic.

In step 216, the processing system may receive feedback regarding the individual's reaction to the content that was sent in step 214. For instance, the feedback may comprise an indication that the individual has viewed the content. In another example, the feedback may comprise an indication that the individual has taken some action with respect to the content (e.g., has used a coupon to make a purchase). In another example, the feedback may comprise an indication that the individual has not viewed and/or take action with respect to the content for some threshold period of time following the sending of the content (e.g., notify the processing system after x minutes if the content has not been viewed). The feedback may be used to update the machine learning model, so that the machine learning model is able to better predict when to send content to the individual in the future.

For instance, reinforcement learning techniques may be applied to learn the mental states under which the individual is most likely to be receptive to receiving content. Reinforcement learning techniques may also be used to adjust the weights of various features in the feature set (e.g., where the weights may be applied by the machine learning model as part of an algorithm for predicting a current mental state). For instance, over time, it may be observed that certain features are more reliable indicators of a particular individual's mental state than other features. As an example, a specific individual's purchasing history may be observed, over time, to have little correlation to the individual's mental state. However, the same individual's heart rate may be observed, over time, to be strongly correlated with the individual's mental state. The features that are observed to be more reliable indicators may be weighted more heavily than the features that are observed to be less reliable indicators.

The method 200 may then return to step 204, and the processing system may continue to acquire data from at least one sensor and to process the data as described in connection with steps 206-216. Thus, the processing system may continuously iterate through the method 200. The iterations may be stopped in response to a request from the individual, in response to the observation of a specific event (e.g., the individual is in the car or at home), or in response to some other criteria.

Thus, examples of the method 200 are able to identify the mental states under which individuals are expected to be most receptive to receiving media content, without relying on intrusive monitoring techniques such as facial or voice analysis techniques. This improves an individual's experience in at least two ways: (1) by minimizing the intrusiveness of the techniques used to monitor the individual's mental state; and (2) by limiting the sending of media content to the times when the individual is expected to be most receptive to the content (and, conversely, by avoiding sending media content at times when the individual is expected to be unreceptive to the content). Thus, individuals are not overwhelmed with a constant stream of content being pushed to their devices at times when they are not emotionally and/or psychologically prepared to receive content.

Although examples of the present disclosure are discussed within the context of advertising and delivering timely commercial content (e.g., advertisements, coupons, etc.), it will be appreciated that the content that may be sent to the individuals' devices may be of a non-commercial nature as well. For instance, the content may comprise reminders of upcoming tasks or appointments that the individuals have scheduled. In other examples, the content may comprise personal messages from family, friends, coworkers, or the like.

Moreover, although the method 200 describes the same processing system as both predicting the individual's current mental state and sending the content to the individual's endpoint device, in other examples these operations could be performed by two separate processing systems. For instance, a first processing system (e.g., of prediction server 115 of FIG. 1, for example) may determine when the best times to send content to the individual are. The first processing system may then send a signal to a separate, second processing system (e.g., of ad server 117 of FIG. 1, for example), where the second processing system sends the content to the individual's endpoint device. Thus, the processing system described above may cooperate with other systems and devices in order to deliver content to an individual at the times that are expected to be most opportune.

Although not expressly specified above, one or more steps of the method 200 may include a storing, displaying and/or outputting step as required for a particular application. In other words, any data, records, fields, and/or intermediate results discussed in the method can be stored, displayed and/or outputted to another device as required for a particular application. Furthermore, operations, steps, or blocks in FIG. 2 that recite a determining operation or involve a decision do not necessarily require that both branches of the determining operation be practiced. In other words, one of the branches of the determining operation can be deemed as an optional step. However, the use of the term “optional step” is intended to only reflect different variations of a particular illustrative embodiment and is not intended to indicate that steps not labelled as optional steps to be deemed to be essential steps. Furthermore, operations, steps or blocks of the above described method(s) can be combined, separated, and/or performed in a different order from that described above, without departing from the examples of the present disclosure.

FIG. 4 depicts a high-level block diagram of a computing device specifically programmed to perform the functions described herein. For example, any one or more components or devices illustrated in FIG. 1 or described in connection with the method 200 may be implemented as the system 400. For instance, a server (such as might be used to perform the method 200) could be implemented as illustrated in FIG. 4.

As depicted in FIG. 4, the system 400 comprises a hardware processor element 402, a memory 404, a module 405 for timing content presentation based on the intended recipient's predicted mental state, and various input/output (I/O) devices 406.

The hardware processor 402 may comprise, for example, a microprocessor, a central processing unit (CPU), or the like. The memory 404 may comprise, for example, random access memory (RAM), read only memory (ROM), a disk drive, an optical drive, a magnetic drive, and/or a Universal Serial Bus (USB) drive. The module 405 for timing content presentation based on the intended recipient's predicted mental state may include circuitry and/or logic for performing special purpose functions relating to the operation of a home gateway or AR server. The input/output devices 406 may include, for example, a camera, a video camera, storage devices (including but not limited to, a tape drive, a floppy drive, a hard disk drive or a compact disk drive), a receiver, a transmitter, a speaker, a display, a speech synthesizer, an output port, and a user input device (such as a keyboard, a keypad, a mouse, and the like), or a sensor.

Although only one processor element is shown, it should be noted that the computer may employ a plurality of processor elements. Furthermore, although only one computer is shown in the Figure, if the method(s) as discussed above is implemented in a distributed or parallel manner for a particular illustrative example, i.e., the steps of the above method(s) or the entire method(s) are implemented across multiple or parallel computers, then the computer of this Figure is intended to represent each of those multiple computers. Furthermore, one or more hardware processors can be utilized in supporting a virtualized or shared computing environment. The virtualized computing environment may support one or more virtual machines representing computers, servers, or other computing devices. In such virtualized virtual machines, hardware components such as hardware processors and computer-readable storage devices may be virtualized or logically represented.

It should be noted that the present disclosure can be implemented in software and/or in a combination of software and hardware, e.g., using application specific integrated circuits (ASIC), a programmable logic array (PLA), including a field-programmable gate array (FPGA), or a state machine deployed on a hardware device, a computer or any other hardware equivalents, e.g., computer readable instructions pertaining to the method(s) discussed above can be used to configure a hardware processor to perform the steps, functions and/or operations of the above disclosed method(s). In one example, instructions and data for the present module or process 405 for timing content presentation based on the intended recipient's predicted mental state (e.g., a software program comprising computer-executable instructions) can be loaded into memory 404 and executed by hardware processor element 402 to implement the steps, functions or operations as discussed above in connection with the example method 200. Furthermore, when a hardware processor executes instructions to perform “operations,” this could include the hardware processor performing the operations directly and/or facilitating, directing, or cooperating with another hardware device or component (e.g., a co-processor and the like) to perform the operations.

The processor executing the computer readable or software instructions relating to the above described method(s) can be perceived as a programmed processor or a specialized processor. As such, the present module 405 for timing content presentation based on the intended recipient's predicted mental state (including associated data structures) of the present disclosure can be stored on a tangible or physical (broadly non-transitory) computer-readable storage device or medium, e.g., volatile memory, non-volatile memory, ROM memory, RAM memory, magnetic or optical drive, device or diskette and the like. More specifically, the computer-readable storage device may comprise any physical devices that provide the ability to store information such as data and/or instructions to be accessed by a processor or a computing device such as a computer or an application server.

While various examples have been described above, it should be understood that they have been presented by way of example only, and not limitation. Thus, the breadth and scope of a preferred example should not be limited by any of the above-described example examples, but should be defined only in accordance with the following claims and their equivalents.

1. A method comprising:
extracting, by a processing system in a telecommunications network, a feature set from data that is collected by at least one sensor, wherein the at least one sensor is monitoring an individual, wherein the feature set comprises at least one feature of the individual, and wherein the feature set excludes features extracted from images of the individual and features extracted from a voice sample of the individual;
predicting, by the processing system, a current mental state of the individual, wherein the current mental state is predicted by providing the feature set as input to a machine learning model; and
based on the current mental state of the individual:
sending, by the processing system, media content to an endpoint device of the individual when the current mental state of the individual indicates that the individual is likely to be receptive to receiving the media content; or
postponing, by the processing system, sending media content to the endpoint device of the individual when the current mental state of the individual indicates that the individual is unlikely to be receptive to receiving the media content.

2. The method of claim 1, wherein the at least one sensor is integrated into an endpoint device of the individual.

3. The method of claim 2, wherein the processing system is integrated into the endpoint device of the individual.

4. The method of claim 1, wherein the at least one feature comprises a health indicator of the individual.

5. The method of claim 4, wherein the health indicator is a blood pressure of the individual.

6. The method of claim 4, wherein the health indicator is a heart rate of the individual.

7. The method of claim 4, wherein the health indicator is a number of steps walked by the individual.

8. The method of claim 1, wherein the at least one feature comprises a context of the individual.

9. The method of claim 8, wherein the context comprises a location of the individual.

10. The method of claim 8, wherein the context comprises information about an application that is currently in use by the individual.

11. The method of claim 1, wherein the at least one feature comprises a purchasing history of the individual.

12. The method of claim 1, wherein the at least one feature comprises a history of interactions of the individual with previously presented media content.

13. The method of claim 1, wherein the at least one feature comprises a preference of the individual.

14. The method of claim 1, wherein the predicting comprises:
constructing, by the processing system, a matrix, wherein the matrix tracks the feature set as measured over a plurality of intervals of time; and
applying, by the processing system, the machine learning model to the matrix, wherein an output of the machine learning model is a prediction of the current mental state.

15. The method of claim 1, wherein the current mental state of the individual indicates that the individual is likely to be receptive to receiving the media content when the current mental state is a happy mental state.

16. The method of claim 1, wherein the current mental state of the individual indicates that the individual is unlikely to be receptive to receiving the media content when the current mental state is an unhappy mental state.

17. (canceled)

18. The method of claim 1, further comprising:
receiving, by the processing system, feedback regarding a reaction of the individual to the media content; and
updating, by the processing system, the machine learning model in response to the feedback.

19. A non-transitory computer-readable medium storing instructions which, when executed by a processing system including at least one processor, cause the processing system to perform operations, the operations comprising:
extracting a feature set from data that is collected by at least one sensor, wherein the at least one sensor is monitoring an individual, wherein the feature set comprises at least one feature of the individual, and wherein the feature set excludes features extracted from images of the individual and features extracted from a voice sample of the individual;
predicting a current mental state of the individual, wherein the current mental state is predicted by providing the feature set as an input to a machine learning model; and
based on the current mental state of the individual:
sending media content to an endpoint device of the individual when the current mental state of the individual indicates that the individual is likely to be receptive to receiving the media content; or
postponing sending media content to the endpoint device of the individual when the current mental state of the individual indicates that the individual is unlikely to be receptive to receiving the media content.

20. A device comprising:
a processing system including at least one processor; and
a computer-readable medium storing instructions which, when executed by the processing system, cause the processing system to perform operations, the operations comprising:
extracting a feature set from data that is collected by at least one sensor, wherein the at least one sensor is monitoring an individual, wherein the feature set comprises at least one feature of the individual, and wherein the feature set excludes features extracted from images of the individual and features extracted from a voice sample of the individual;
predicting a current mental state of the individual, wherein the current mental state is predicted by providing the feature set as an input to a machine learning model; and
based on the current mental state of the individual:
sending media content to an endpoint device of the individual when the current mental state of the individual indicates that the individual is likely to be receptive to receiving the media content; or
postponing sending media content to the endpoint device of the individual when the current mental state of the individual indicates that the individual is unlikely to be receptive to receiving the media content.

21. The device of claim 20, wherein the predicting comprises:
constructing a matrix, wherein the matrix tracks the feature set as measured over a plurality of intervals of time; and
applying the machine learning model to the matrix, wherein an output of the machine learning model is a prediction of the current mental state.
